(19) United States
(12) Patent Application Publication
(10) Pub. No.: US 2011/0097800 A1
(43) Pub. Date: Apr. 28, 2011

(54) METHOD AND APPARATUS FOR RETAINING AND RECIRCULATING CELLS

(75) Inventors: Joerg Kauling, Koln (DE); Björn Frahm, Erkrath (DE); Marc Jenne, Leverkusen (DE); Helmut Brod, Koln (DE); Stephan Kirchner, Wuppertal (DE); Arndt Braun, Remscheid (DE)

(73) Assignee: Bayer Technology Services Gmbh, Leverkusen (DE)

(21) Appl. No.: 12/997,281

(22) PCT Filed: Jun. 10, 2009
(86) PCT No.: PCT/EP2009/004200

§ 371 Date: Jan. 11, 2011

(30) Foreign Application Priority Data
Jun. 20, 2008 (DE) 10 2008 029 307.5

(51) Int. Cl. C12N 5/07; C12M 3/02; B29C 53/58; B32B 7/08

(52) U.S. Cl. 435/393; 435/293.1; 156/195; 156/91

(57) ABSTRACT

The invention relates to a device and a method for retaining and recirculating cells in a vessel through which flow passes continuously or batchwise.

In addition, the invention relates to a method of producing a device by which cells can be retained and recirculated in a vessel through which flow passes continuously or batchwise.

This application is a 371 of PCT/EP2009/004200, filed Jun. 10, 2009, which claims foreign priority benefit under 35 U.S.C. §119 of the German Patent Application No. 10 2008 029 307.5 filed Jun. 20, 2008.

The invention relates to a device for retaining and recirculating cells in a vessel through which flow passes continuously or batchwise, which device can be operated inside or outside a bioreactor. The invention further relates to a method of retaining and recirculating cells inside or outside a bioreactor. In addition the invention relates to a method of producing a device by which cells can be retained and recirculated in a vessel through which flow passes continuously or batchwise.

Culturing animal and plant cells is of great importance in the production of biologically active substances and pharmaceutically active products. In particular, the culturing of cells which is frequently carried out in a nutrient medium in free suspension is demanding, because the cells, in contrast to microorganisms, are very sensitive with respect to mechanical shear stress and insufficient supply with nutrients.

Usually, animal and plant cell lines are cultured batchwise. This has the disadvantage that optimal supply of the cells can be achieved only with difficulty as a result of the constantly changing substrate, product and biomass concentrations. At the end of the fermentation, in addition, by-products accumulate, e.g. components of dead cells which must usually be removed with great effort in the later workup. For the said reasons, but in particular in the production of unstable products which can be damaged, e.g. by proteolytic attacks, therefore continuously operated bioreactors are preferably used.

With continuous bioreactors, high cell densities and high productivity which is associated therewith may be achieved if the following requirements are met:
sufficient and low-shear supply of the cells with substrates, in particular dissolved oxygen,
sufficient disposal of the carbon dioxide produced in respiration,
an effective, low-shear, blockage-proof cell retention system for building up high cell concentrations
long-term stability (sterility, hydrodynamics) of the bioreactor.

In addition to the continuous mode of operation, a bioreactor having an efficient cell retention system can be used, e.g. also for culturing precultures having particularly high cell densities. Then, the cell retention system is used discontinuously in order to take off cell culture supernatant virtually free from biomass. Thereafter the preculture reactor can again be filled with fresh nutrient medium and the culture may be brought in this manner to higher cell densities than with simple batchwise operation.

Frequently, for the low-shear supply of the cells with dissolved oxygen, bubble-free gas introduction by means of membranes is used. For example, membranes may be developed as flexible tubes on cylindrical basket stators (Henzler, H.-J., Kauling, J., Oxygenation of cell cultures, Bioprocess Engineering, 9, 1993, 61-75, EP A 0172478, EP A 0240560). To make space for large mass transfer surfaces, the flexible tubes are placed tightly next to one another with the smallest possible spacing.

With the aid of low-shear, radially transporting agitator elements such as blade or anchor agitators, flow passes in a radial direction through the concentrically arranged flexible tube membranes in order to reduce the liquid-side mass transport resistance.

A further possibility of supplying the cells with dissolved oxygen is bubble gas sparging with oxygen-containing gases. The use of coarse-bubble gas sparging and dispersion of the bubbles using an agitator element, however, are restricted to low cell densities because of the low specific phase interface area of coarse bubbles and the associated low mass transport. In addition, the viability of the cells suffers because of the mechanical shear stress which accompanies the dispersion of bubbles using an agitator element at high performance ranges which are not customary for cell culture (WO 03/020919 A2).

For this reason, in recent years, fine-bubble gas sparging for supplying the cells with dissolved oxygen has become established (Nienow, A. W., Reactor Engineering in Large Scale Animal Cell Culture, Cytotechnology, 50, 1-3, 2006, 9-33, Varley, J., Birch, J., Reactor design for large scale suspension animal cell culture, Cytotechnology, 29, 3, 2004, 177-205).

Fine-bubble gas sparging is produced using special sinter bodies made of metallic and ceramic materials, filter plates or laser-perforated plates, wherein the pores or holes are generally smaller than 15 μm. At low superficial gas velocities of below 0.5 m/h, very fine gas bubbles are generated which, in the media usually used in cell culture, have a low tendency to coalescence. The agitator merely has the task of distributing the fine gas bubbles in the bioreactor, but not their generation.

In order that a high cell density (>20 million living cells per millilitre) can be achieved in a continuously operated bioreactor, in addition efficient retention of the cells is necessary. The required degree of retention depends in this case on the growth rate of the cells and the perfusion rate q/V (media throughput q per bioreactor volume V).

In the past, different cell retention systems have been proposed for continuously operated bioreactors, which are mostly arranged outside the bioreactor. The reason for this is the easy accessibility of the cell retention system for maintenance and cleaning purposes.

To keep damage to the cells, in particular owing to inadequate oxygen supply and carbon dioxide removal, outside the bioreactor as low as possible, cell retention systems having small working volumes and, associated therewith, short residence times of the cells, are desirable.

In addition to membrane filters, apparatuses which operate by the principle of cross flow filtration using fixed and movable membranes, special centrifuges and gravity separators are used.

In the case of cell retention using membrane filters, deposits and/or foulings are observed which prevent reliable and maintenance-free long-time operation. The depositions can be reduced if the flow over the membrane surfaces is rapid. However, this requirement counteracts the basic precondition of low-shear cell culture fermentation.

Special low-shear centrifuges have been developed for separating off cells in the centrifugal field. However, these centrifuges only operate over a few weeks without maintenance work. The exchange of centrifuge elements which is required during the maintenance work increases the risk of insterility.

A further possibility for separating off the cells from the cell culture broth is using gravity separators. The gravity separators which are predominantly used in cell culture are settling vessels and inclined channel separators. Compared with simple settling vessels, inclined channel separators have the advantage of a considerably smaller volume. One publication (Henzler, H.-J., Chemie-Technik, 1, 1992, 3) describes cell retention in inclined channel separators which can be operated in countercurrent flow, cross flow and cocurrent flow. The channel cross section through which flow passes can be provided with plates or tubes. The patent publications U.S. Pat. No. 5,817,505 and EP 0 699 101 B1 claim the use of inclined channel separators for retaining cells in countercurrent flow separators. In WO2003020919 A2, inter alia, countercurrent flow and cross flow separators, and also combinations with various preseparators (e.g. hydrocyclones) for retaining cells are described.

The inclined channel separators are connected via an external circuit to the bioreactor. For this flexible tube lines and pumps are required, the use of which increases the complexity of the plant and thereby the risk of failure. In addition, the shear stress of the cells is increased.

To reduce the metabolic activity and the caking of cells in a gravity separator, cooling the cell culture broth on its path to the gravity separator is proposed. Reduced metabolic activity at low temperature is certainly advantageous in the case of relatively long residence of the cells outside the bioreactor. The development of temperature and density gradients in the interior of the gravity separator which can lead to the efficiency-reducing flow phenomenon of free convection is avoided by restricting the cooling temperature.

Bioreactors are also described in which the cell retention system is arranged inside the bioreactor. EP 0 227 774 B1 describes a continuously operated fermentation kettle in which the cells are retained inside an airlift loop reactor. The airlift loop flow passes the cell suspension around the internal flow-calmed settling zone which is formed by vertical partition walls. The cells which are deposited in the settling zone are recirculated to the agitated cell suspension, while a culture supernatant is taken off at the top of the settling zone. The disadvantage of the described vertically acting retention appliance, however, is the difficulty of scaling it up. This leads to a disproportional enlargement of the separator volume compared with the fermentation zone. The consequence is high residence times of the cells in inadequately suppliable separators, with the consequence of reduced productivity of the reactor system.

Therefore, in the light of the prior art, the object is to provide an efficient method for retaining and recirculating animal and plant cells in a continuously or batchwise operated method which takes into account the sensitivity of cells with respect to mechanical shear stress and adequate supply of the cells with nutrients, which meets the maintenance, cleaning and sterility requirements of the pharmaceutical industry and use of which decreases the complexity and risk of failure.

The invention therefore relates to a device for retaining and recirculating cells in a vessel through which flow passes which comprises a multiplicity of adjacently arranged channels, wherein the channels form an upright hollow cylinder and are inclined at an angle β between 10° and 60° to the longitudinal axis of the hollow cylinder.

The vessel through which flow passes can be a bioreactor or a vessel connected to a bioreactor for cell retention and cell recirculation.

The flow can pass through the vessel continuously or batchwise, preferably it passes through continuously.

The channels are open at the lower end. At the upper end they lead into a shared annular space which has at least one line via which the harvest stream can be transported from the vessel.

The cells and cell culture solution are separated in the channels. As a result of the continuously take off of the harvest stream from the bioreactor, cell culture solution and cells are drawn into the channels. The cells sediment within the channels which are arranged at an incline and slide, as in classic inclined channel separators, in countercurrent flow to the inflowing harvest stream, out of the channels again and thereby remain in the vessel. The cell culture solution which is separated from the cells is transported by the channels into the annular space above the channels and finally out of the vessel.

The channels have a polygonal, elliptical or round cross section. The inclined channel plates which are known from the prior art have a rectangular profile. The separation surface area for the sedimenting cells is designed to be planar in rectangular profiles. A square channel having the cross sectional width d has a greater separation surface area than a round channel having the same diameter d. Surprisingly, however, it has been found that the efficiency in retention and recirculation of cells in round channels having a diameter d, despite the lower separation surface area, corresponds to the efficiency of rectangular channels having the cross sectional width d. A possible explanation would be that the friction between the sedimented cells and the channel inner wall in the case of a round cross section is lower owing to the lower contact area, and thus the cells can more readily slide down. Also, it is conceivable that avalanche effects play a role. Since the sedimenting cells in a round channel cross section increasingly come to lie one above the other owing to an additional compacting process directed towards the lowest point on the vertical axis, they pull one another more readily than in a rectangular cross section. This ultimately leads to a reduction of the cells present in the separator and thereby to an increase in the free flow cross sections.

Preferably, the channels therefore have a cross section decreasing towards their lower side. Particularly preferably, the channel cross section on the lower side has a semicircular or elliptical profile. The use of channels having a cross section decreasing towards the lower side instead of straight plates according to the prior art leads to a significantly accelerated slipping of the cells, in such a manner that a possible exhaustion of dissolved oxygen in the channels can be counteracted. In a preferred embodiment of the device according to the invention, the channels have a round cross section.

The dimensioning of the channels (number, diameter, length) depends in each case on the type of cells to be retained, the size of the bioreactor and the throughput.

The required separation surface area A_{erf} is given by the sedimentation velocity ws, the perfusion rate q/V (media throughput q per bioreactor volume V) and the bioreactor volume according to equation 1. Efficiency η takes into account the reduction in performance of inclined channel separators compared with vertical separators (equation 2).

The theoretical separation surface area Aₜₕ for rectangular and cylindrical cross sections can be determined to an approximation from equation 3 and equation 4 according to the approaches published in the literature (H.-J. Binder, Sedimentation aus Ein- und Mehrkornsuspensionen in schrag stehenden, laminar durchstromten Kreis- und Rechteckrohren [Sedimentation from single- and multigrain suspensions in inclined laminar-flow circular and rectangular tubes], Dissertation Berlin, 1980):

$$\begin{matrix}{A}_{erf} = \frac{Perfusion rate \cdot V}{ws} & ( equation 1 ) \\ {A}_{th} = \frac{{A}_{erf}}{η} & ( equation 2 ) \\ rectangle \text{:} {A}_{th} \approx Z \cdot sin ( β ) \cdot d \cdot L & ( equation 3 ) \\ cylinder \text{:} {A}_{th} \approx \frac{3 \cdot π}{16} \cdot Z \cdot sin ( β ) \cdot d \cdot L & ( equation 4 )\end{matrix}$$

Here, Z is the number of channels, β the angle by which the channels are tilted with respect to the direction of gravity, d the internal diameter and L the length of the channels. π is the mathematical constant of a circle (π=3.14159 . . . ).

The angle β depends on the settling and slipping behaviour of the cells and is preferably 10°≦β≦60°. In a preferred embodiment the angle β is between 15° and 45°, particularly preferably between 25° and 35°. For improvement of the slipping behaviour, the device can be vibrated by suitable means, for example pneumatic or electric vibrators. At high volumetric concentration or cell densities>20 million cells/millilitre and restricted vibration possibility, angles of 20°≦β35° are particularly preferred.

It is conceivable to vary the angle over the length of the channel.

The channel width d (maximum cross sectional width, in the case of a round profile the diameter of the channel) is preferably d≧3 mm, in order to prevent blockage of the channels. In a preferred embodiment, channels having a channel width of 3 mm to 100 mm, preferably of 5 mm to 20 mm, particularly preferably of 5 mm, are used in order to reliably prevent firstly blocked states, but secondly to keep as low as possible the volume ratio that reduces the space-time yield of separator and bioreactor space.

In the dimensioning of the channel length, maintaining laminar flow conditions (Re<2300; Re=Reynolds number) must be taken into consideration. On installation into a container, the channel length depends on the vertically available container inner dimension, and/or on the fill levels to be achieved in the reactors. Short channel lengths, owing to the reduced pressure drops, can lead to distribution problems which, in particular during take off of the harvest solution from the upper annular space, can require a complex distribution device for reducing the take off velocities. The dynamic pressure at the take off site should in this case be at least 5- to 10-times lower than the pressure drop in the channels. In this respect channel lengths from 0.1 m may be considered as industrially achievable, whereas channel lengths of 0.2 m to 5 m are preferred and/or of 0.4 m to 2 m are particularly preferred.

The device according to the invention comprises 2 to 10⁶ channels, preferably 10 to 100 000, particularly preferably 100 to 10 000 channels.

The shell of the upright hollow cylinder formed by the channels comprises one or more layers of channels. Preferably it comprises 1 to 100 layers, particularly preferably—in particular in the case of internal installation into a bioreactor—1 to 10 layers. The layers can be arranged in a ring shape or spirally around one another. The layers can be connected to a stator which offers mechanical support.

The cylinder, on its installation into the bioreactor, preferably has a height of 30% to 95%, particularly preferably of 60%-90% of the fill height of the bioreactor. This installation permits a directed flow past the cylinder. The flow past the cylinder offers the advantage that the cylindrical bioreactor wall can be additionally utilized, e.g., for heat exchange or for accommodating sensors on installation of the separator device. The circulating flow in addition induces or promotes the suspension of particles. Expedient bottom shapes of the bioreactor have rounded corners or are constructed as dished or round bottoms. In the case of a central gas feed close to the bottom, the sedimenting particles, e.g. the microbial or eukaryotic cells, are transported by the circulating flow to the bottom centre, where they are taken up and resuspended by the upwards-directed gas-treatment induced flow, if appropriate with the aid of agitator systems. Under the said installation conditions, favourable cylinder diameters are 50-85% of the reactor diameter, depending on the separator surface area to be accommodated and/or the number of the ring-shaped or spiral channel layers to be mounted. It must be ensured in this case that the annular surface situated between bioreactor wall and stator can occupy 5-300%, particularly preferably 100%, of the cylinder cross section. In this manner it is ensured that a circulating flow can be induced with high efficiency without excessively great friction losses. The required separator surface areas depend on the sedimentation properties of the cells and also on the sought-after perfusion rates and cell concentrations. Preferred perfusion rates are in the range of 0.2-40 l/day, particularly preferably between 0.5 and 20 l/day. Preferred separator surface areas per bioreactor volume, depending on sedimentation properties of the cells (depending on concentration, size and agglomeration tendency of the cells) are in the range between 0.1 and 100 m²/m³, particularly preferably between 2 and 20 m²/m³.

The outward and inward directed shell surfaces of the cylinder are preferably sealed, in order to prevent the penetration of cells into the channel intermediate spaces and thereby prevent fouling.

A cylinder in the context of the present description is bounded by two parallel planar surfaces (base and top surface) and a shell or cylinder surface which is formed by parallel lines. It is formed by displacing a planar guide curve along a straight line which does not lie in this plane. Accordingly, the cylinder formed by the channels can have various shapes. It can be, e.g., a circular cylinder, a cylinder having an elliptical base surface or a prism, i.e. a cylinder having a polygon (triangle, rectangle, pentagon, . . . ) as base surface. Other shapes are also conceivable, such as, e.g., the arrangement of the channels in the shape of a truncated cone. Preferably, it is a cylinder having a circular or elliptical base surface. The cylinder has an internal channel (hollow cylinder) which runs in parallel to the shell surface and preferably has the same cross sectional shape as the base surface.

Preferably, tubes or flexible tubes are used as channels. Materials which come into consideration are, e.g., plastics or metals. Preferably, use is made of plastics which are known to those skilled in the art such as Teflon, silicone rubber (hereinafter called silicone for short), polyethylene or polypropylene. Preferably, use is made of materials for the tubes or flexible tubes which have low tendency to adhesion of biomass. A particularly highly suitable material is silicone, since it can be processed very well with a quality sufficient for pharmaceutical processes. In addition, it is oxygen-permeable, so that an oxygen supply can be achieved, to a certain degree, even within the channels. For this, the outer space around the channels can be flushed with oxygen-containing gas. This is fed by means of a gas feed line and outlet line into the intermediate space of the channels, i.e. between the upper and lower channel holders.

The device according to the invention as a whole, or parts of the device according to the invention can preferably be constructed as disposable articles to avoid cleaning problems.

In a preferred embodiment of the device according to the invention, silicone tubes are used as channels. The silicone tubes are preferably joined to one another to form mats and are coiled onto a cylindrical stator in one or more layers until the desired separation surface area is achieved. The mats of flexible tubes at an incline are preferably constructed as a disposable element which reduces to a minimum the expenditure for providing a retention system purified according to pharmaceutical principles.

In a preferred method of producing the device according to the invention, as channel, a flexible tube or tube is coiled over a cylinder. The individual coils in this case are preferably closely adjacent to one another. A plurality of layers of flexible tube or tube can be coiled one over the other, if a plurality of layers of channels are required in the device. The individual coils are preferably joined to one another mechanically, e.g. by gluing. The length of the later channels corresponds to the circumference of the coils around the cylinder. Ignoring the extension of the channels, the channel length L is given by the circumference U of the cylinder to an approximation by equation 5 (π=mathematical constant of a circle).

$$L = \frac{U}{π} \qquad ( equation 5 )$$

The number of coils gives the number Z of the later channels.

Subsequently, the coiled tube or coiled flexible tube is cut through transversely to the coils. This proceeds in a spiral around the cylinder (see, e.g., FIG. 3). In this case the gradient of the spirals gives the later angle β of the channels which are at an incline. The result is a mat of one or more layers of channels which are at an incline (see, e.g., FIG. 4). The mat can be joined at the inclined ends, so that a sock is formed (hollow cylinder). This can, as required, be pulled onto a support body (stator) (see, e.g. FIG. 5). While the underside of the channels remains open, the top side is joined to a holder in such a manner that above the channels an annular space is formed into which, during operation of the device, the liquid streams which flow through the individual channels meet.

It is advisable to seal the inside and outside of the hollow cylinder from the exterior in order to prevent cell culture solution and cells from penetrating into the intermediate spaces between the channels and causing fouling. Likewise, the intermediate spaces between the channels should be hermetically closed on the lower side and top side of the hollow cylinder in order to prevent the penetration of cell culture solution and cells. By this special design feature of the hermetically sealed channel exterior space, it is possible to flush this space with oxygen-enriched gas. By using oxygen-permeable materials for the channels, preferably silicone, the cells which are retained in the separator space can be supplied with oxygen.

According to a further preferred production method, the device according to the invention is formed from a profiled film (see, e.g., FIG. 6). A profiled film preferably comprises a smooth side and a side having a sequence of ridges and grooves at constant intervals. Channels form when the film is coiled in a spiral or shell shape in one or more layers, e.g. onto a stator. In this case the grooves, toward the open side, are each closed by the smooth side of an adjacent layer or by the wall of the stator.

The geometry of the channels is established by the ratio of ridge height hs to channel width b. Technical achievable hs/b ratios are between 0.33 and 5 according to the properties (deformability, elasticity, deep-draw capacity). It must be noted in this case that both dimensions hs and b should each be greater than or equal to 3 mm, or preferably greater than or equal to 5 mm Preferred hs/b ratios are 0.5 to 3. The ridge widths bs are determined by the mechanical stability of the film material. The ridge widths bs should be as small as possible in order to enable high shearing areas per unit of separator volume. At the same time, they should not be selected to be too small, in order to permit non-positive joining to the lower layer without shape change.

In the case of the spiral coiling, ramp-like connections and closures to the mats are required in order to achieve axial sealing between fermenter space and take off space at the transitions.

In the case of a shell-shaped coiling, for increasing the stability of the individual layers, it can be logical to change, from layer to layer, the direction of the channels alternately between a positive and negative angle of incidence. In one embodiment of the device according to the invention, mats having channels arranged at an incline are therefore arranged in the shape of a shell, wherein each shell is formed by a mat which is shaped from a hollow cylinder and the individual mats can be rotated with respect to their adjacent mats by 180° to one of the longitudinal axes of the mat. In a preferred embodiment, the mat of every second shell is rotated by 180°.

The profiled film can be obtained by shaping immediately on film production or by (e.g. adhesive) joining of an embossed, hot- or cold-shaped film to a smooth film. The material properties of the embossed and smooth films can be optimally adapted, i.e. by selection of a suitable material known to those skilled in the art and having a corresponding surface quality, to their differing functionality (good sliding properties and shape stability of the embossed film, good density properties of the smooth film).

Since in the production method with use of film channels a device results with which a flushing of gas around the channels and mass transport which is inducible thereby would only be achievable with great expenditure, devices of this type are preferably provided for use outside a bioreactor.

The methods described permit simple and inexpensive production of a device for retaining and recirculating cells. By choice of the cylinder around which the tube or the flexible tube is coiled, the number of coils, the coil spacing, the number of layers and the gradient of the cut spirals, the geometry of the later device may be simply and exactly established. Likewise, the geometry of the later device may be simply and exactly established by the choice of perforated film and the number of coils (layers).

The methods described permit, in particular, the inexpensive production of disposable elements, use of which reduces to a minimum the expenditure of providing a retention system purified to pharmaceutical principles.

The device according to the invention may be connected and operated simply within or outside a bioreactor. The connection, operation and maintenance are problem-free. Design of the device according to the invention or parts of the device according to the invention as disposable elements eliminates cleaning problems.

The use of the device according to the invention inside a bioreactor reduces the formation of temperature and density gradients inside the settling zone, in such a manner that unwanted convection streams and an associated adverse effect on the efficiency of cell retention can reliably be avoided. Also, the arrangement inside the bioreactor reduces the complexity and the risk of failure of the overall installation compared with, for example, the external arrangement of an inclined channel plate separator.

In a preferred embodiment, the separator installation is therefore used inside a bioreactor. There it divides the fermentation zone into two regions, into a cylindrical inner space and an annular outer space.

Preferably, the device according to the invention is combined with means for generating a circulating flow. The circulating flow transports the cell culture solution together with the cells contained therein through the cylindrical inner space along the outer surface of the cylindrical device through the annular outer space and again through the cylindrical inner space. Suitable means for generating the circulating flow are, for example, mechanical agitators or gas-treatment systems. Particularly preferably, the circulating flow is achieved by means of a system for treatment with fine gas bubbles, in such a manner that via the treatment with gas bubbles, not only oxygen input can be effected, but also a natural circulation between both fermentation regions can be induced, without an additional agitator element having to be used. In this manner the highly integrated reactor described leads to numerous advantages for cell culture fermentation:
The circulation reactor is low-shear and possesses excellent mixing behaviour and gas-supply behaviour.
As a result of the loop flow, despite the separator internal, the reactor wall can in addition be used for heat exchange, so that integration into existing fermentation units is ensured.
Installation of a scalable (i.e. proportional enlargement of the separator volume with the bioreactor volume) retention surface area within the bioreactor abolishes the coupling of bioreactor and separator downstream of autoclaving which is associated with an increased infection risk. In addition, all cell-transporting pumps and also numerous external flexible tube lines can be dispensed with. The consequence of this is, inter alia, abolition of the temperature change between cooled separator zone and reheated fermentation zone, a reduction of the shear stress and an increase in process robustness.
The cells which slip back into the bioreactor in countercurrent flow to the harvest stream from the separator are, by means of the circulating flow, transported back into the well supplied part region of the bioreactor promptly and without additional pumps in a particularly low-shear manner.

In a particularly preferred embodiment, a bioreactor in combination with the device according to the invention is constructed as an airlift bioreactor (cf. e.g. EP 0 227 774 B1) in which the gas, such as, for example air, is introduced into an upwards-directed part of the bioreactor, also known as a riser in the art. Preferably, treatment with fine gas bubbles takes place, wherein the use of surfactants for avoiding foam and also for keeping the cells away from high-shear gaseous interfaces can be helpful. The riser is connected at its top and bottom ends with the top and bottom ends of a further, upwards-directed part of the bioreactor, known in the art as a downcomer. A widespread variant of the essentially cylindrical airlift bioreactor contains a centrally arranged cylindrical guide tube which divides the airlift bioreactor into a lift part (riser) within the guide tube and a sink part (downcomer) in the annular space between the guide tube and the container outer wall of the airlift bioreactor. The lift part can equally well be found in the annular space between the guide tube and the container outer wall and the sink part within the guide tube. The feed of, for example, oxygen-enriched gas, at the lower end of the riser reduces the mean density of the suspension culture in the riser which leads to an upwards-directed liquid flow in the riser which consequently replaces the liquid contents of the downcomer which in turn flows back to the bottom end of the riser. In this manner a liquid circulation is generated which adequately mixes the suspension culture and keeps the cells suspended, i.e. in free suspension. The advantage of a bioreactor agitated in such a manner is that with adequate supply of the cells with oxygen dissolved in the nutrient medium and adequate disposal of the carbon dioxide resulting from respiration, no moving parts such as a mechanical agitator are necessary. The cross sectional surface areas of the riser and downcomer are essentially the same.

In a particularly preferred embodiment, the device according to the invention forms a guide tube between downcomer and riser of a continuously operated airlift bioreactor. By suspension culture of comparable temperature flowing round the device according to the invention, flow phenomena of free convection are avoided.

A further preferred embodiment is the spatially separated arrangement of fermentation zone and separator zone, i.e. the device according to the invention is connected externally to the bioreactor. Supply of the separator is ensured by at least two pumps, preferably low-shear peristaltic pumps. The pumps enable the cell culture solution to be taken off from the bioreactor space, to be fed after cooling via a heat exchanger to the settling apparatus, take off of the harvest stream from the settling apparatus and transport of the concentrate stream back to the bioreactor.

The cooling device required for cooling the fermentation medium can be integrated into the housing of the separator which is preferably constructed as a disposable element and can thereby likewise be constructed as a disposable element, in such a manner that the cleaning requirements needed for this essential device are also dispensed with.

A perfusion reactor consisting of bioreactor and internal or external retention appliance can be operated in a known manner. Nutrient medium is supplied continuously and low-cell cell culture supernatant is removed continuously. The perfusion reactor can be operated at high perfusion rates q/V (media throughput q per bioreactor volume V) if this is biologically meaningful, and sufficient separator surface area is provided.

Likewise, a bioreactor having an internal or external retention device can be operated in such a manner that a culture is first allowed to develop batchwise. When the medium is consumed to the extent that no significant build up of biomass is any longer possible, culture supernatant which is virtually free of biomass is taken off via the internal or external retention device. The space obtained in the bioreactor can then be utilized in order to feed fresh nutrient medium, as a result of which further growth and thereby higher overall biomass productivity are enabled. This method is suggested as an example of precultures with which very large bioreactors shall be inoculated, since it can increase the productivity of existing preculture reactors.

The bioreactor can be used for culturing cells which grow in vitro and in free suspension or on microcarriers. The preferred cells include protozoa and also adhesive and non-adhesive eukaryotic cells of human, animal or plant origin which are capable, e.g. by genetic modification, of producing special active pharmaceutical agents such as viruses, proteins, enzymes, antibodies or diagnostic structures. Particularly preferably, use is made of cells which are suitable for pharmaceutical high performance production, for example ciliates, insect cells, Baby Hamster Kidney (BHK) cells, Chinese Hamster Ovary (CHO) cells, HKB cells (resulting from the fusion of human HEK 293 cell line with the human Burkitt lymphoma cell line 2B8), or hybridoma cells.

The present invention further relates to a method of retaining and recirculating cells in a vessel through which flow passes. Fresh and/or treated medium is fed to the vessel continuously or batchwise and exhausted medium is removed by the device for retaining and recirculating cells situated within the vessel. The device consists of a multiplicity of channels arranged at an incline which form an upright hollow cylinder and are inclined at an angle β between 10° and 60°, preferably at an angle β between 15° and 45°, particularly preferably at an angle between 25° and 35°, to the longitudinal axis of the hollow cylinder.

In the channels which are arranged at an incline, preferably a flow velocity prevails which permits the maintenance of laminar flow states in accordance with Re<2300, whereby an efficiency-decreasing resuspension of the deposited cells against the field of gravity is avoided.

The Reynolds number Re can be calculated in accordance with equation 6 from the flow velocity w averaged over the cross section, the kinematic viscosity v of the flowing medium and the inner diameter d of one channel:

Re=(w·d/v) (equation 6)

In this case, a lower flow velocity prevails at the channel inner walls than in the channel centres. The cells sediment in the channels and slide on the bottom side of the channels against the direction of flow to the bottom channel ends. Preferably, in the vessel, a circulation flow prevails which entrains the cells at the bottom channel ends and distributes them in the vessel. The circulation flow preferably proceeds as loop flow around the inner and outer surfaces of the upright hollow cylinder of channels. The method according to the invention is therefore preferably combined with a circulation flow in the vessel through which flow passes continuously. The cell culture solution freed from the cells is transported by the channels into an annular space which is arranged above the channels and finally out of the vessel.

The method according to the invention can be carried out inside a bioreactor. In this case the cells are retained inside the bioreactor. In the bioreactor and in the separator zone, uniform temperatures occur, such that convection flows in the separator are excluded. Under these conditions the cells, however, on the other hand are also able to continue their metabolism and respire oxygen. By flushing the outer space of the device with an oxygen-enriched gas, the oxygen consumption can be counteracted and its biological consequences alleviated. In this case the oxygen diffuses through the oxygen-permeable channels walls into the flow channel which can be considered to be relatively well mixed, at least in the lower channel cross sections, that is in the region of high cell concentration, by the feed and sedimentation processes proceeding intensely there. The cells which are slipping down and are situated in these regions have only a short residence time in the system such that a short-time deficit of the optimum supply concentration of the cells can generally also be withstood without damage. The supply of the cells in the top channel cross sections is considerably more critical owing to the in some cases very long residence times of 10-45 minutes, such that an oxygen supply in these regions can prove to be particularly helpful.

The method according to the invention can also be carried out outside a bioreactor. For this, the cell culture solution is transported with cells out of the bioreactor into a vessel in which a multiplicity of channels arranged at an incline is arranged in the form of an upright hollow cylinder. In this vessel the cells and cell culture solution in which the mixture is transported through the channels are separated, where the cells sediment, slide against the direction of flow to the end of the channels and finally pass into a settling zone from which they can be transported again back to the bioreactor. Preferably, the cells are cooled in the external vessel in order to retard metabolism and thereby counteract undersupply of the cells which reduces productivity. In a cooled suspension, an oxygen supply of the sedimenting cells by flushing the channels from the outside is not absolutely necessary. Usually, cooling the cell culture solution to the ambient temperature of the separators is completely sufficient, so that in addition to the desired metabolic effect, convection flows are safely avoided.

The method according to the invention permits the effective retention and recirculation of cells in a vessel through which flow passes continuously. In the course of the retention and recirculation, only moderate shear forces act on the cells which are generally tolerated well by the cells. The cells in the channels are kept at fermentation temperature or a reduced temperature level and they are supplied with nutrients. Mass transfer may be optimized, if required, by additional gas-treatment of the intermediate spaces of the channels or the outsides of the channels.

Likewise, the method permits retention and recirculation of cells in a vessel in which, by continuous or discontinuous media exchange, higher cell densities can be achieved than in a batch culture process without media exchange. In this manner the method can advantageously be used in order to increase the productivity of preculture reactors, the biomass of which is used to inoculate very large batch operated bioreactors. In addition, the method can extend the possibilities of use of fed-batch fermenters in which the biomass is collected during the product harvest in order to inoculate a new fermenter in what is termed repeated fed-batch mode.

### BRIEF DESCRIPTION OF THE DRAWINGS

Hereinafter, exemplary embodiments of the invention will be described in more detail with reference to drawings, without restricting the invention thereto.

FIG. 1 shows schematically an embodiment of the device according to the invention. Channels (10) having a round cross section form a hollow circular cylinder (20). The shell of the circular cylinder comprises one layer of channels which are arranged at an incline. The channels are tilted at an angle β to the longitudinal axis of the circular cylinder. In operation, the longitudinal axis is preferably identical to the direction of gravity. FIG. 1(a) side view; FIG. 1(b) view of a cross section along the dashed line through the cylinder (20) in FIG. 1(a) from the top or bottom.

FIG. 2 shows schematically the conditions of cell separation in a channel (10) having a round cross section. The channel (10) is charged (1) from the bottom with the cell suspension. The harvest stream (2) is taken off at the top end of the channel. The cell retentate (3) sediments on the lower side of the channel and slides against the direction of flow to the lower channel end.

FIG. 3 shows schematically a method for producing a mat from channels which are arranged at an incline. A tube or flexible tube (200) is coiled over a cylinder (300). In this case, to accommodate the largest possible separator surface areas on a small space, the coils are close to one another. The coils are preferably mechanically joined to one another in order to ensure that the channel orientation is retained when it is later cut. For this the channels can be joined to one another at points or surfaces directly or via the cylinder outer surface by means of a carrier layer, e.g. a woven fabric or nonwoven. A preferred joining proceeds via adhesion. Suitable adhesives are the adhesive components matched to the material and surface properties of the channels and known to those skilled in the art. In the case of a silicone tube, preferably silicone adhesives are used which are available on the market in the required FDA quality classes. During the adhesion care must be taken to ensure that the flexible tubes are not joined to one another so as to seal in order to permit flow of gas around the gas-permeable channels. After the adhesion, the flexible tube mat is cut along the dashed cutting line (210) running spirally around the cylinder and is taken off from the cylinder. The result is a mat of channels arranged at an incline, as shown in FIG. 4.

FIG. 4 shows schematically a mat (220) of channels (10) having a round cross section which are arranged at an angle β to a theoretical line along the channel lower sides. Such a mat (220) is obtainable by a method shown in FIG. 3 and explained in the description of FIG. 3. The inclined longitudinal sides (230, 231) can be joined to one another in order to obtain one layer of separator channels of the device according to the invention for retaining and recirculating cells. For enlargement of the surface area, a plurality of layers can be adhered to one another along their longitudinal sides (230, 231) in a shell-shape. It is likewise possible to coil the mat to give a plurality of layers one above the other in a spiral shape. In both cases here, a sock (hollow cylinder (20)) is formed having one or more layers of channels (10) which are arranged at an incline (see, e.g., FIG. 1, FIG. 5). This can be pulled onto a mechanical support (stator).

FIG. 5 shows schematically the lower side of a device according to the invention in perspective view. One layer of channels having a round cross section (10) is arranged around a tubular stator (5). The channels are tilted at an angle β to the longitudinal axis of the hollow cylinder (20).

FIG. 6(a) shows an example of a profiled film (250). The film comprises a sequence of grooves (251) and ridges (252) at a constant spacing.

FIG. 6(b) shows a cross section through the film of FIG. 6(a) along the line B-B. The film can be cut to form mats. One such mat appears in cross section by way of example just as the film cross section in FIG. 6(b). The mats can be placed flat one above the other and are joined to one another, wherein the ridges (252) of a mat are joined to the smooth lower side of a mat lying there above such that the grooves form channels which are closed along the ridges. The joined mats can then be shaped to form an upright hollow cylinder and joined at the sides in a similar manner to the example of FIG. 4. Likewise it is possible to coil the profiled film around a stator in one or more layers. In this case the grooves (251) facing the open side are in each case sealed with an adjacent layer or with the wall of the stator, forming channels.

In the case of a film of the type of FIGS. 6(a) and (b), channels having a rectangular cross sectional profile are formed. Likewise it is conceivable to use films having other profiles. In FIG. 6(c), e.g., a film having a semicircular groove profile is shown. Correspondingly other channel geometries are produced thereby.

FIG. 7(a) shows by way of example a profiled film as a composite of an embossed film (260) and a smooth sealing film (265) which are joined by means of adhesive (270).

FIG. 7(b) shows the joining of three profiled films (250), wherein in each case the smooth lower side of a film is joined to the ridges of the film beneath, thereby giving a multiplicity of adjacently arranged channels (10).

FIG. 8 shows one design of the device according to the invention in cross section perpendicular to the longitudinal axis of the hollow cylinder formed by channels (10-1, 10-2). The hollow cylinder having a circular base comprises two layers which are arranged in the shape of a ring on channels having round cross section ((10-1)=channel in the first layer, (10-2)=channel in the second layer). The hollow cylinder is preferably sealed on the outside and inside by a shell (outside (13), inside (14)), in order that no cells pass into the intermediate spaces (15) between the channels (10-1, 10-2) and cause fouling.

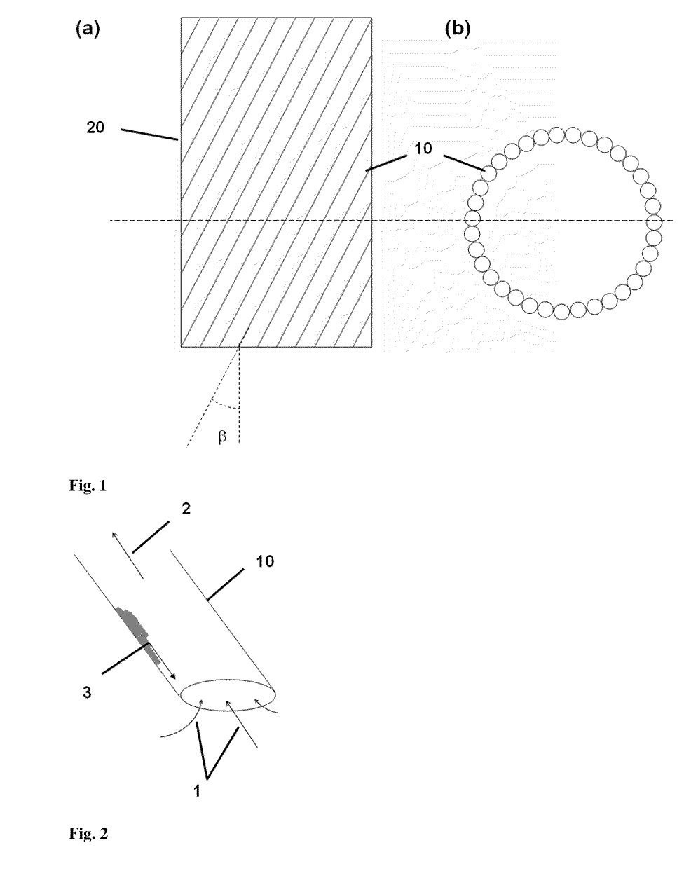

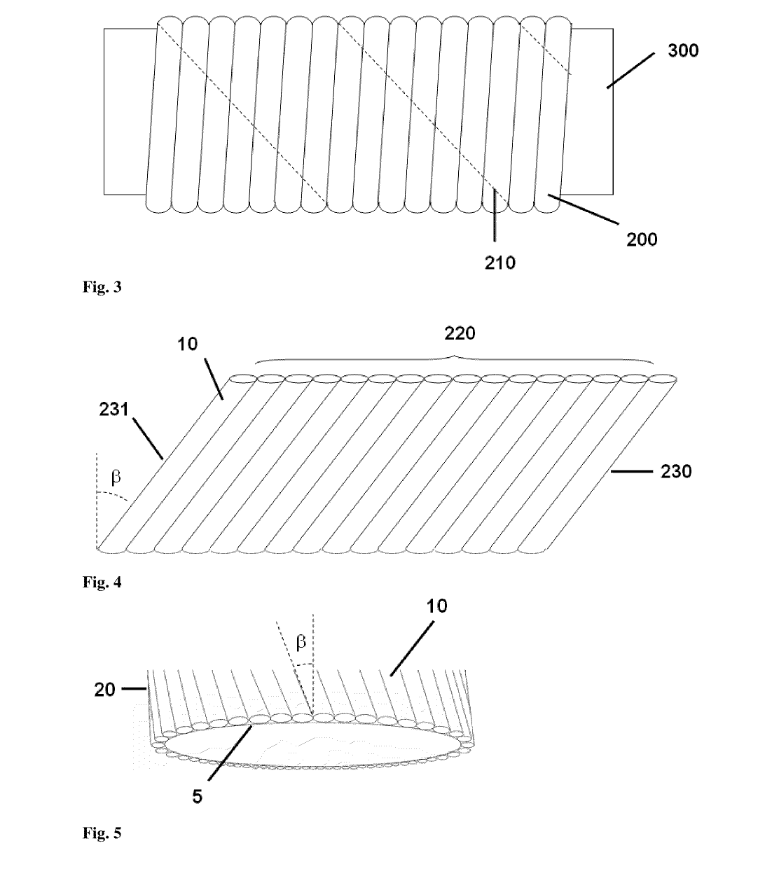

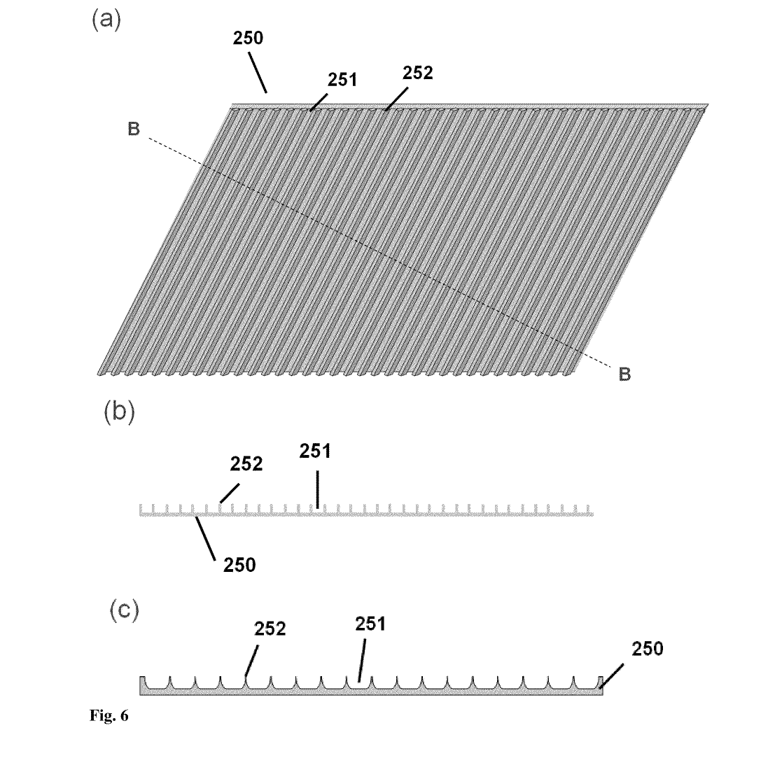

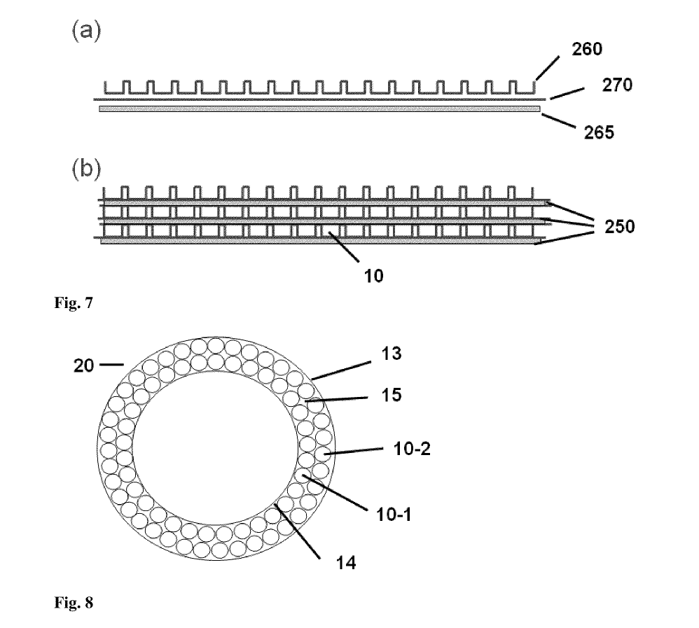

In FIG. 9, the installation of the cell retention system (400) is shown by way of example in a gas-bubble aerated bioreactor (100) in cross section. The cell retention system comprises channels (10) arranged at an incline which are arranged in a plurality of layers around a flexible tube stator (5). In the drawing the channels (10) are drawn vertically for graphical reasons. However, according to the invention they are tilted to the longitudinal axis of the flexible tube stator (5). The preferably microfine gas bubbles generated via the gas distributor (40) ensure a natural circulation between the central reactor zone (51) in the example shown which is gas treated and through which flow passes upwards, and the non-gas-treated reactor zone (52) close to the wall through which flow passes downwards. In this manner, in addition to the oxygen transport, good mixing of the reactor is ensured by gas-liquid means. The harvest stream (2) is taken off after the cell separation in the cell retention system (400) at the top port of the bioreactor (100). The particles which are separated off in the cell retention system (400) are transported back into the gas-treated reactor centre with the circulation flow. Sedimentation in the reactor is at the same time effectively prevented by the circulation flow. The exhaust gas is removed via ports (42) at the top of the reactor. The gas treatment of the outer space around the separator flexible tubes proceeds by means of the gas feed lines and outlet lines (21) and (22) which are connected to the cell retention system (400). Further feed lines and outlet lines to/from the loop reactor (100) are the media supply (30) and the temperature-control medium feed (61) and outlet lines (62) into a jacket (60) for the temperature control of the bioreactor (100).

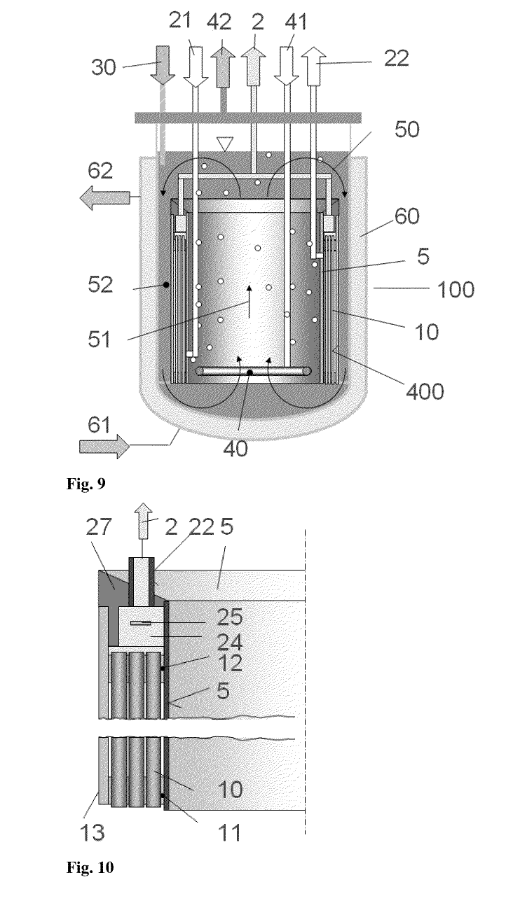

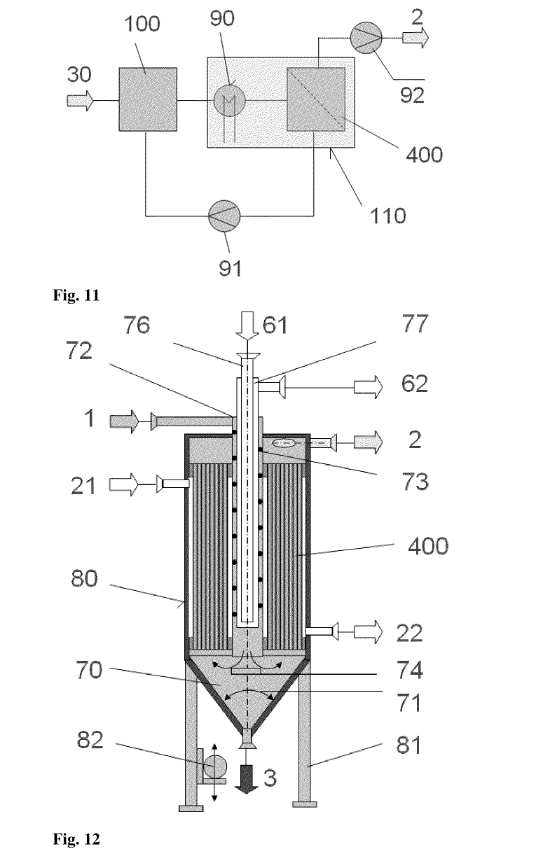

FIG. 10 shows the arrangement of the channels (10) in the form of flexible tubes which are coiled to form a plurality of layers (flexible tube mats) and are held in a lower bonding site (11) and an upper bonding site (12). In the drawing the channels (10) are drawn vertically for graphical reasons. However, according to the invention they are tilted to the longitudinal axis of the flexible tube stator (5). The material forming the bonding sites (11) and (12) is, e.g. a flexible adhesive material known to those skilled in the art, e.g. preferably based on silicone, which tightly encloses the flexible tubes which are preferably made of silicone and which form the channels (10) and which adhesive material provides smooth sealing surfaces in both radial directions to the inside and outside. By means of the sealing surfaces, a sealing action against the flexible tube stator (5) and also against the shell (13) can be effected. The shell (13) is sealed via the collar projecting over the flexible tube mats against the top element (27) which is joined to the flexible tube stator (5). The sealed construction shown in FIG. 8 ensures that the space around the flexible tubes is not filled with liquid. In a preferred arrangement, this intermediate space between the flexible tubes is flushed with an oxygen-enriched gas in order to improve the oxygen supply of the sedimented cells during the sedimentation process. The top part (27) is joined to the flexible tube stator (5) via a slope (28) which is intended to prevent deposition of cells. The harvest ports (22) which are welded into the slope (28) open out into an annular space (24) just above the channels (10). In order to ensure, in the case of a restricted number of harvest ports, a favourable liquid distribution, e.g. tangential flow guiding, fractal flow distribution or the installation of baffle plates (25) is advisable.

In FIG. 11, the classic method schematic with an external arrangement of the cell retention system (400) integrated into the separator installation (110) is shown. In order to reduce the respiratory activity of the cells in the bioreactor sequence, the temperature thereof is decreased to a lower level as directly as possible after take off in a cooling device (90). In this manner the cells are prevented from dwelling too long in an oxygen-limited state in the cell retention system (400), which could damage the cells physiologically. In the example shown, the separator (110) consists of a cell retention system (400) and the integrated cooling device (90). The liquid streams between bioreactor (100) and separator (110) are adjusted via the low-shear pumps (91) and (92). Other circuit arrangements, e.g. the positioning of one of the two pumps (91) and (92) in the bioreactor outlet would likewise be conceivable.

In FIG. 12 the separator (110) which is integrated into the housing (80) and comprises cell retention system (400) and cooling device (90) is shown. The cell culture solution (1) is introduced cooled into the separator via the downpipe (72), which may be deaerated if appropriate, below the cell retention system (400). The cooling proceeds along the riser pipe (77) in which the cooling liquid ascends in countercurrent flow to the downwards-flowing cell culture solution. Particularly good heat transport and therefore a compact construction of the cooler are ensured by the low wall thickness of the riser pipe (77), a high velocity of the cooling medium in the gap between riser pipe and immersed pipe (76) and (77) and also a spiral shaped flow internal for the cell culture solution between the downpipe (72) and riser pipe (76). Before entry into the cone it is advisable to reduce the velocity of the cell culture solution in order to prevent the concentrated cell mass which is already deposited from being swirled up again. In order to ensure large entry cross sections, the cooling device should not be moved down right to the intake region. Depending on velocity, the additional installation of a baffle plate (74) is advisable. After the cell sediment has slipped back from the cell retention system (400) into the cone (70), the cell retentate (3) can be removed at the cone tip. Cone angles of 20°-70° have proved to be usable. In order to avoid too great a height of the structure, a cone angle as small as possible should be preferred, but which can reliably avoid blocked states. Therefore, cone angles of 40°-60° are among the preferred embodiments. In the case of adequate vibration, a cone angle of 45° is particularly preferred. For the oxygen supply of the sedimented cell mass, a gas feed (21) and gas take off (22) can be supplied from the outside via connection ports which are welded into the housing (80). For the gas treatment the use of channels (10) made up of flexible tubes is required.

### REFERENCE SIGNS

1 Cell culture solution

2 Harvest

3 Cell retentate

5 Flexible tube stator

10 Inclined channel

11 Lower bonding site

12 Upper bonding site

13 Shell outside

14 Shell inside

15 Intermediate space

20 Cylinder

21 Gas feed

22 Gas take off

24 Ring channel

25 Baffle plate

27 Top part

28 Slide surface

30 Media feed

40 Gas distributor

41 Gas supply

42 Gas removal

50 Loop flow

51 Upwards flow

52 Downwards flow

60 Jacket

61 Feed of temperature-control medium

62 Outlet of temperature-control medium

70 Cone

71 Cone angle

72 Downpipe

73 Spiral

74 Baffle plate

76 Immersed pipe

77 Riser pipe

80 Housing

81 Frame

82 Vibrator

90 Cooler

91 Return pump

92 Harvest pump

100 Bioreactor

110 Integrated separator

200 Tube/flexible tube

210 Intersection line

220 Mat of tubes/flexible tubes

230 Longitudinal side of the mat

231 Longitudinal side of the mat

250 Profiled film

251 Groove

252 Ridge

260 Embossed film

265 Sealing film

270 Adhesive

300 Cylinder

400 Cell retention system

1. Device for retaining and recirculating cells in a vessel through which flow passes continuously or batchwise, comprising a multiplicity of adjacently arranged channels, wherein the channels form an upright hollow cylinder and are inclined at an angle β between +10° and +60° to the longitudinal axis of the hollow cylinder.

2. Device according to claim 1, wherein the channels have a cross section with a width decreasing towards their lower side.

3. Device according to claim 1, wherein the shell of the hollow cylinder comprises 1 to 100 layers of channels.

4. Device according to claim 1, wherein the channels have an inner diameter of 3 mm-30 mm.

5. Device according to claim 1, wherein the channels are formed from flexible tubes.

6. Device according to claim 1, additionally comprising means for treating the outer surfaces and/or intermediate spaces of the channels with gas.

7. Device according to claim 1, additionally comprising means for generating a circulating flow through the hollow cylinder and along the outer surface of the hollow cylinder.

8. Device according to claim 1, wherein a cooling device is integrated.

9. A continuously operated airlift bioreactor comprising a device according to claim 1.

10. Method of retaining and recirculating cells in a vessel through which flow passes continuously or batchwise, comprising transporting a cell-containing medium through a multiplicity of adjacently arranged channels in which the cells sediment and from which the cells slide out again, wherein the channels form an upright hollow cylinder and are inclined at an angle β between 10° and 60° to the longitudinal axis of the hollow cylinder.

11. Method according to claim 10, which further comprises effecting, by gas treatment, and/or by stiffing, a circulating flow of the medium through the hollow cylinder and along its outer surface.

12. Method according to claim 10, which further comprises gas treating the outer surface of the channels and/or the intermediate spaces between the channels.

13. Method of producing a device for retaining and recirculating cells, comprising shaping a film or mat comprising adjacently arranged channels or grooves by spiral or shell-type coiling to form a hollow cylinder having one or more layers of channels which are at an inclination to the longitudinal axis of the hollow cylinder.

14. Method according to claim 13, wherein the mat comprising adjacently arranged channels is formed by the means that a tube or flexible tube is coiled around a cylinder in 1 to 100 layers, adjacent coils are mechanically connected to one another and the composite of coils is divided along an intersection line running spirally around the cylinder.

15. Method according to claim 13, wherein the film comprising adjacently arranged grooves is formed by the means that an embossed or hot- or cold-shaped film is joined to a smooth film to form a film composite.
